(19) United States
(12) Patent Application Publication
(10) Pub. No.: US 2007/0297618 A1
(43) Pub. Date: Dec. 27, 2007

(54) System and method for controlling headphones

(75) Inventors: Mikko Nurmi, Tampere (FI); John Patrick Wong, Vancouver (CA); Michael Ahokas, Melbourne (AU); Risto Kontio, Burnaby (FI)

Correspondence Address: FOLEY & LARDNER LLP, P.O. BOX 80278, SAN DIEGO, CA, 92138-0278, US

(73) Assignee: Nokia Corporation

(21) Appl. No.: 11/474,777

(22) Filed: Jun. 26, 2006

(51) Int. Cl. A61F 11/06

(52) U.S. Cl. 381/71.6

(57) ABSTRACT

A system for controlling an electronic device. The system includes an earpiece for distributing sound to a user. The earpiece has a sensor which is adapted to sense when the state of the earpiece changes from use to non-use and from non-use to use. The earpiece is adapted to be in communication with an electronic device to provide state information from the sensor to the electronic device so that the electronic device can respond, such as by ceasing to play a song when the earpiece is removed by a user.

### FIELD OF THE INVENTION

The present invention relates generally to electronic devices having video or audio outputs. More particularly, the present invention relates to personal mobile devices that utilize headphones.

### BACKGROUND OF THE INVENTION

It has become increasingly common to listen to music, watch television or recorded video media, talk on the phone, or even play video games using a mobile device. Such devices typically include an audio component. As such, numerous mobile audio devices utilize an earpiece or similar device to transmit audio content to a user. Typically, such devices include a mass storage device, input keys, a battery, and software for controlling the hardware. For example, mobile music players typically use an earpiece connected to the mobile music player via a wire for transmitting the music signal. Another common mobile device used with an earpiece is a mobile telephone. In fact, multi-purpose devices often combine several functions into a single item to allow a user to utilize an earpiece to listen to music, audio tracks accompanying video content, telephone conversations, and video games.

One of the challenges of such mobile audio devices is the service life or operation time, with battery life being the primary limiting factor. Currently, the typical life of batteries (or discharge time if a rechargeable battery) is reduced by the operation of the device when it is not in use, such as where the user forgets to turn off the device or where the device is accidentally activated.

In addition to operational concerns such as battery life, it is occasionally desirable to interrupt the audio from the mobile audio device. Mobile audio devices have become so ubiquitous that they are used in situations where the user still needs to interact with their environment. For example, mobile audio users may travel to and from work on mass transit, with an earpiece in place for using the mobile telephone. However, where the user wishes to engage in a conversation or is asked a question by another passenger, it is highly desirable to be able to stop the audio in order to enable the user to hear the other person.

Current designs allow a user of a mobile audio device to control the audio content either using controls on the mobile audio device itself or using a wired earpiece cord that contains controls. However, mobile audio device usage is often limited by the inability to easily control device usage based on whether a user is using the device. Often this is due to drainage of the batteries cause by operation of the device when the user is not using it. Thus, there is a need for a system and method for provide communication between an earpiece and a mobile audio device regarding the state of use of the mobile audio device.

### SUMMARY OF THE INVENTION

The present invention provides systems and methods for controlling an electronic device based on the usage state of an earpiece. An earpiece in accordance with the present invention includes a sensor for detecting the usage state of the earpiece. In one embodiment, temperature is used to determine if the earpiece is in use. In another embodiment, a capacitive sensor is used to determine if the earpiece is in use.

The earpiece is adapted to be in communication with an electronic device, such as a mobile audio device. When the sensor detects a change in the state of the earpiece, a signal communicates this change to the mobile audio device. The mobile audio device then modifies its function based on the signal. In one embodiment, when the earpiece is removed, i.e. changes to a not-in-use state, the mobile audio device pauses the playback of the current audio content. When the earpiece is replaced, i.e. changes to an in-use state, the mobile audio device resumes playback of the audio content.

These and other objects, advantages, and features of the invention, together with the organization and manner of operation thereof, will become apparent from the following detailed description when taken in conjunction with the accompanying drawings, wherein like elements have like numerals throughout the several drawings described below.

### BRIEF DESCRIPTION OF THE DRAWINGS

FIG. 1 is an illustration of an earpiece and mobile audio device in accordance with the principle of the present invention;

FIG. 2 is a flow diagram showing one process for implementation or the present invention;

FIG. 3 is an illustration of one embodiment of the present invention having a temperature sensor;

FIG. 4A is an external view of an earbud-style earpiece having a temperature sensor; FIG. 4B is an internal view of the earbud of FIG. 4A;

FIG. 5 is an illustration of an earbud style earpiece having a capacitance sensor;

FIG. 6 is an external view of a headset-style earpiece with an exploded view of a mechanical switch type sensor;

FIG. 7 is an illustration of a mobile telephone of the present invention;

FIG. 8 is a schematic representation of the telephone circuitry of the mobile telephone of FIG. 7; and

FIG. 9 is an illustration of an embodiment of the present invention having an earpiece in communication with a mobile telephone.

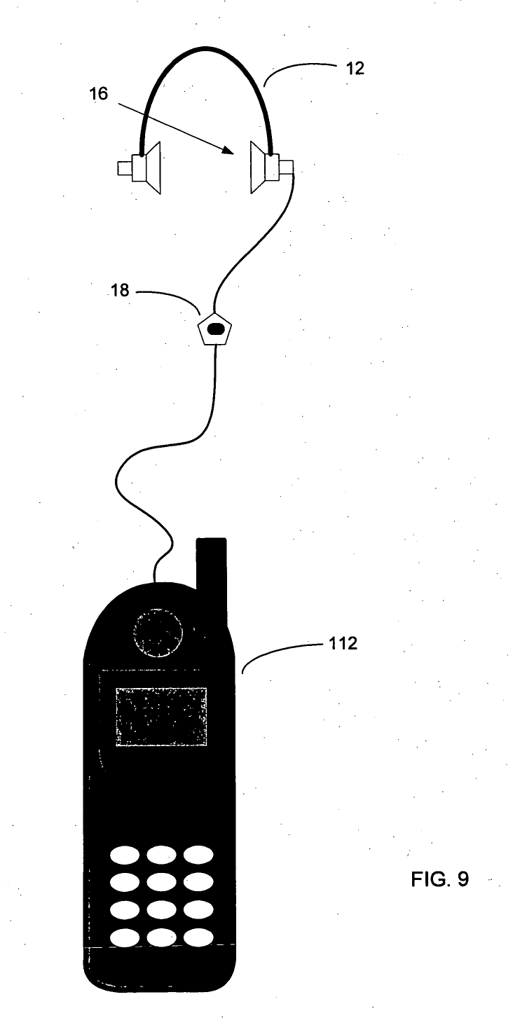

### DETAILED DESCRIPTION OF THE PREFERRED EMBODIMENTS

The present invention relates to a system and method for controlling the audio output of an audio device. Most audio devices, particularly mobile audio devices, use earpieces to provide a user with audio content via an audio feed such as playing a song. Audio content may include audio files in various storage formats, over-the-air broadcasts, and Internet or network streaming feeds. Often, the user removes the earpieces and would prefer the audio content to stop, either so that the user can engage in another activity, such as a conversation, or because the user no longer wishes to use the audio device. In general, the present invention provides an earpiece with a sensor capable of detecting the usage state of the earpiece to determine when the audio device should be stopped.

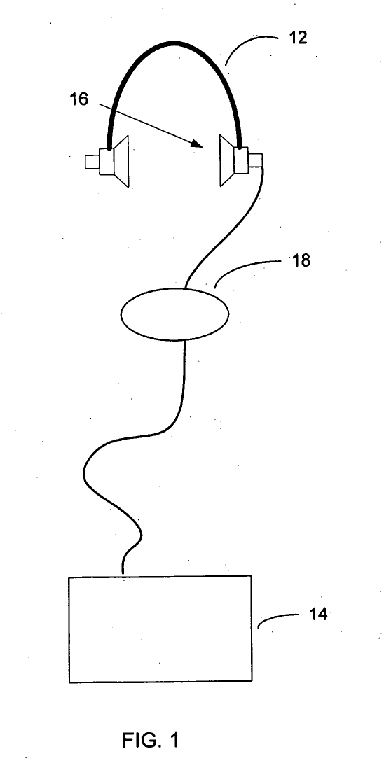

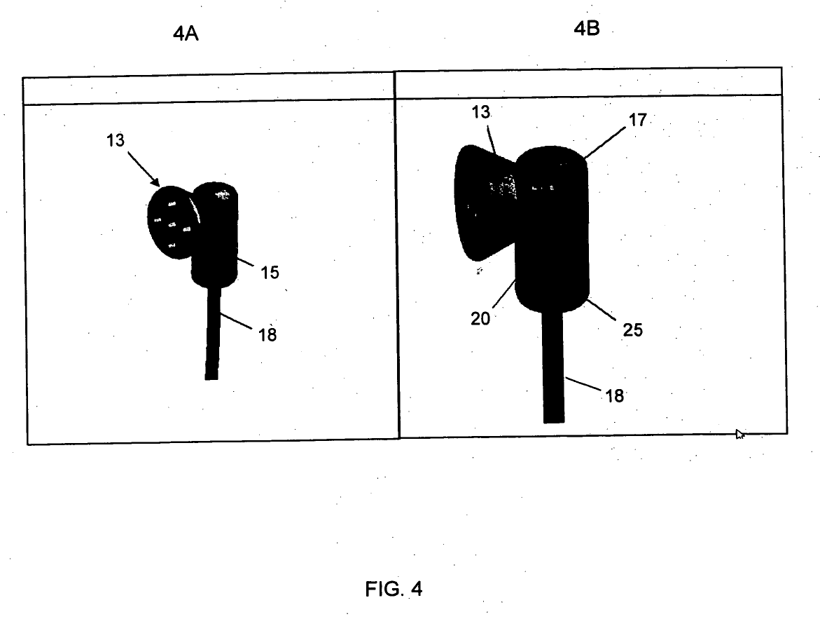

In the embodiment shown in FIG. 1, the system includes an earpiece 12 having a communication mechanism 18 in communication with a mobile audio device 14. The earpiece 12 may be of any of the various types of earpieces, such as earbuds or headphones, designed for use in a single ear or both ears, or include a microphone such as a telephone headset. The communication mechanism 18 enables communication, in one embodiment two-way communication, between the earpiece 12 and the mobile audio device 14, such as either wired or wireless. In general, the earpiece 12 of the present invention includes at least one speaker 13 of FIGS. 4, 5, and 6 at least partially disposed within a housing 15 of FIGS. 4, 5, and 6. The earpiece 12 includes a sensor 16 for detecting info such as when the earpiece 12 is in use. The sensor 16 is in communication with the mobile audio device, preferably by the same mechanism as the mobile audio device sends audio content to the earpieces, e.g. a wired connection or a wireless connection.

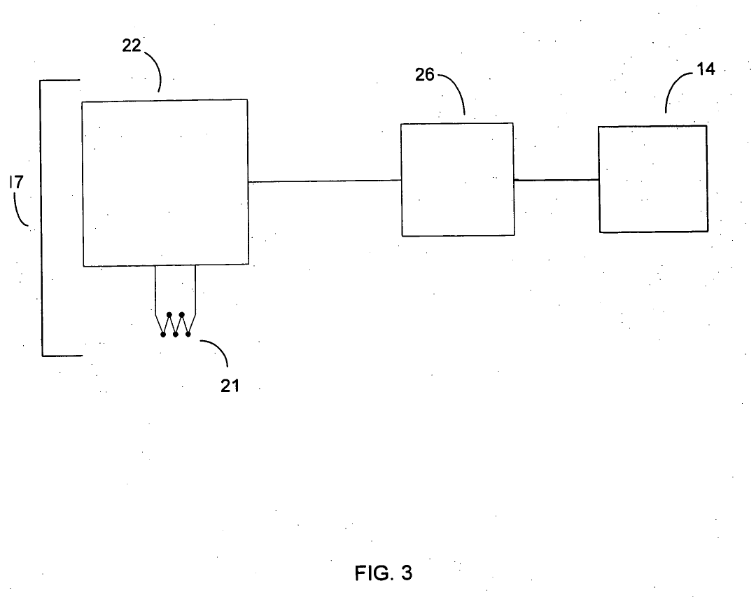

In accordance with the principles of the present invention, the sensor 16 is capable of detecting the usage or wear state of the earpiece 12, i.e. whether the earpiece 12 is in use (e.g., being worn) or not in use (e.g., removed). In an exemplary embodiment, the sensor is in communication with a printed circuit board 20 of FIG. 4B. In one embodiment, the sensor 12 outputs a signal to an applied control interface (ACI) 26 of FIG. 3. The ACI is used for communication between the earpiece 12 and the mobile electronic device 14.

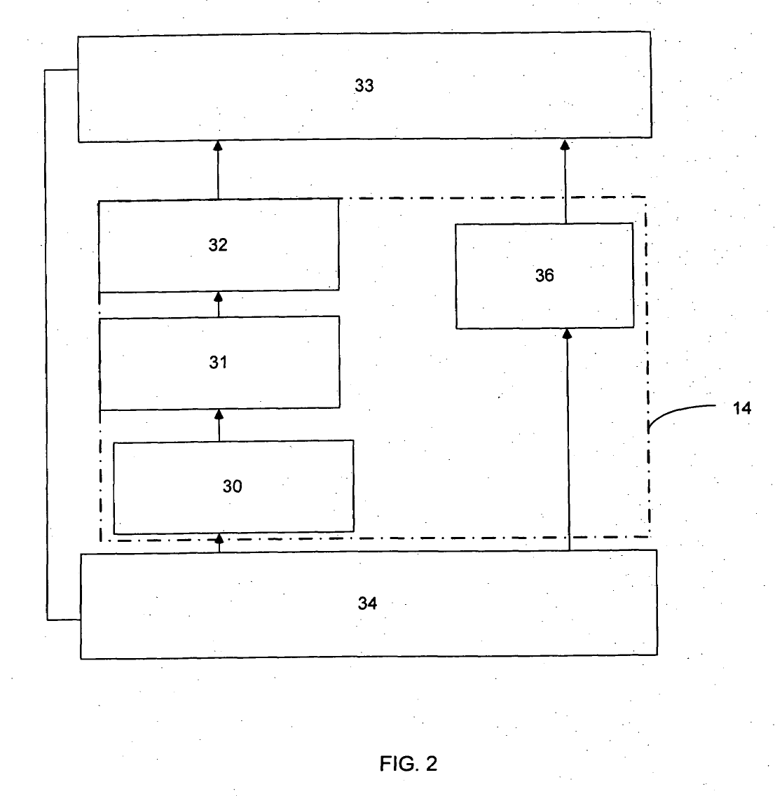

In the embodiment illustrated in FIG. 2, the method of controlling the mobile electronic device 14 is interrupt based. The mobile audio device 14 receives an interrupt from the earpiece when the user is not wearing the headset. During play, at a digital signal-processing step 30, the mobile audio device receives or reads from memory a digital signal including audio content. At a digital signal-processing step 31, this digital signal is processed and sent to a digital/analog converter. At the conversion step 32, the digital/analog converter converts the digital signal to analog, and this analog signal is sent to the earpiece 12. During the analog signal reception step 33, the earpiece receives the signal and converts the signal to sound waves. At the user sense step 34, the earpiece constantly monitors the state of the earpiece to determine if the earpiece is in use. If the earpiece is in use, then the mobile audio device continues to process the digital signal for transmission to the earpiece and conversion to sound. If the earpiece is not in use, then at an interrupt step 36, the earpiece 12 transmits a non-use interrupt to the mobile audio device 14. The non-use interrupt causes the mobile audio device 14 to change its state to stop playing the audio content.

In the exemplary embodiment shown in FIG. 3, the sensor includes a temperature sensor 17. In one embodiment, the temperature sensor 17 is in contact with a printed circuit board 20 and includes a temperature-sensing element 21 and temperature sensor circuitry 22. The temperature sensor 17 is adapted to be in communication with the mobile audio device 14 via an ACI 26. The mobile audio device 14 checks the ACI for an interrupt to determine if the mobile audio device needs to change modes. For example, the mobile audio device 14 may change from standby to play when the earpiece is placed in use, or from play to standby when the earpiece is no longer in use. In one embodiment, the temperature range indicating use comprises a range around human body temperature (37 degrees Celsius), for example 35 to 39 degrees Celsius. In one embodiment, the temperature sensor is only active during the playing of an audio content.

In the embodiment of FIGS. 4A and 4B, the earpiece 12 includes, in addition to the temperature sensor 17, a motion sensor or accelerometer 25. While the temperature sensor 17 is adapted to determine if the earpiece 12 is in use, situations may arise where the earpiece 12 is not in use while the temperature sensor 17 detects a temperature within the ranged determined to correspond to in-use. For example, the earpiece 12 and mobile audio device 14 may be left in a hot car or sitting in the sun were a high temperature is detected but the user is not using the mobile audio device 14. Thus, it is sometimes desirable to determine by another mechanism if a user is using the mobile audio device 14. In an exemplary embodiment, the mobile audio device 14 is controlled by both the temperature sensor 17 and the motion sensor 25. In this embodiment, the mobile audio device 14 will change mode to a standby mode when an interrupt is received, reflecting a temperature outside of the desired range and motion below a desired amount.

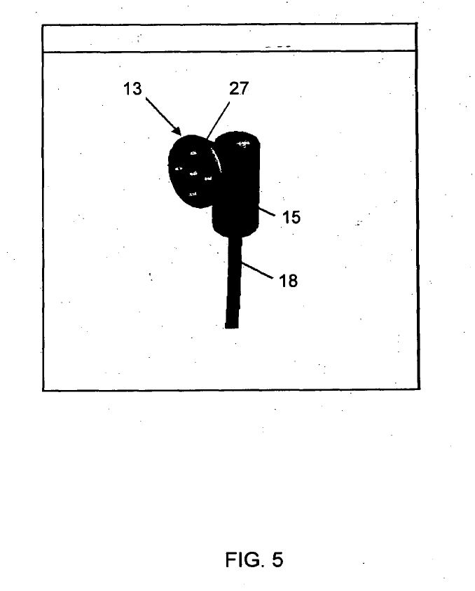

In yet another embodiment shown in FIG. 5, the sensor 16 includes a capacitive sensor 27 that detects contact with a user's skin. In one embodiment, the earpiece 12 is an earbud style headphone, which includes a capacitive sensor 27 for contact with the inner portion of a user's ear.

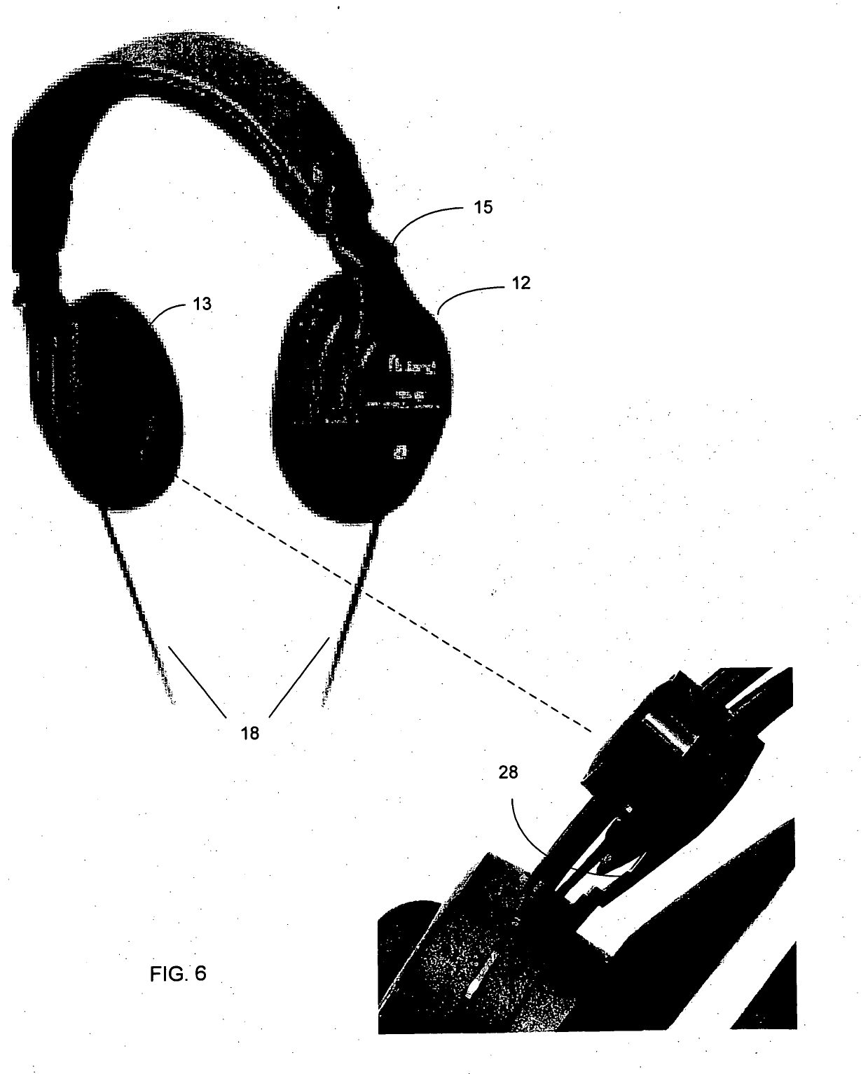

In still another embodiment shown in FIG. 6, the sensor 16 includes a simple mechanical switch 28 that detects whether the earpiece 12 is in use. In one embodiment, the earpiece 12 is a non-earbud headphone that has a semi-rigid structure. The switch 28 is turned on when the user wears the earpiece due the flexing of the earpiece 12 when in use.

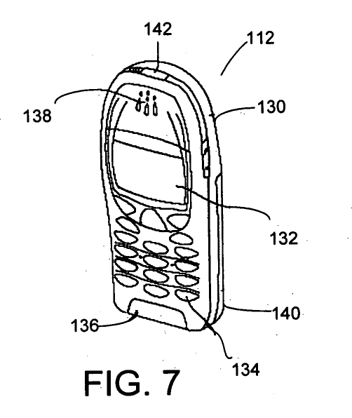

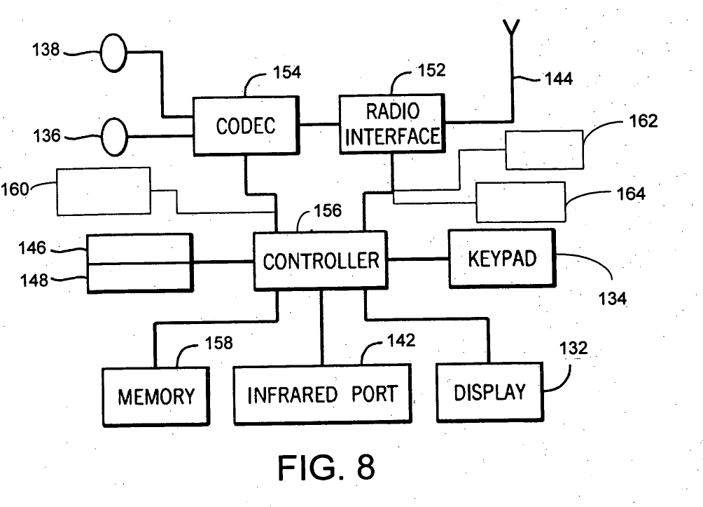

In one embodiment, the mobile audio device comprises a mobile telephone. FIG. 7 shows one representative mobile telephone 112 within which the present invention may be implemented. It should be understood, however, that the present invention is not intended to be limited to one particular type of mobile telephone 112 or other electronic device. FIG. 8 depicts a mobile telephone having digital audio functionality in accordance with the principles of the present invention. The mobile telephone 112 of FIG. 8 includes a housing 130, a display 132 in the form of a liquid crystal display (LCD), a keypad 134, a microphone 136, an ear-piece 138, a battery 140, an infrared port 142, an antenna 144, a smart card 146, in the form of a universal integrated circuit card (UICC) according to one embodiment of the invention, a card reader 148, radio interface circuitry 152, codec circuitry 154, a controller 156 and a memory 158. In one embodiment, an application control interface (ACI) 160 is provided. In a further embodiment, the mobile telephone 112 includes a digital radio 162 and a digital media player 164 for playback of audio files. Individual circuits and elements are all of a type well known in the art, for example in the Nokia range of mobile telephones. Other types of electronic devices with which the present invention may be incorporated can include, but are not limited to, personal digital assistants (PDAs), integrated messaging devices (IMDs), and notebook computers.

Various control systems are possible depending upon the functionality of the mobile audio device 14. For example and in one embodiment, the mobile audio device 14 would stop playing the audio content, such as a song, when the earpiece 14 is removed, and the mobile audio device 14 would resume the audio content at the same spot when the earpiece 12 is placed back in use. In one embodiment, an energy conservation step is performed after a predetermined time following a change in earpiece 12 status to unused. In an exemplary embodiment, the mobile audio device 14 powers down after a predetermined time after a user removes the earpiece 12.

The present invention is described in the general context of method steps, which may be implemented in one embodiment by a program product including computer-executable instructions, such as program code, executed by computers in networked environments.

Generally, program modules include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represent examples of corresponding acts for implementing the functions described in such steps.

Software and web implementations of the present invention could be accomplished with standard programming techniques, with rule based logic, and other logic to accomplish the various database searching steps, correlation steps, comparison steps and decision steps. It should also be noted that the words “component” and “module” as used herein, and in the claims, is intended to encompass implementations using one or more lines of software code, and/or hardware implementations, and/or equipment for receiving manual inputs.

The foregoing description of embodiments of the present invention have been presented for purposes of illustration and description. It is not intended to be exhaustive or to limit the present invention to the precise form disclosed, and modifications and variations are possible in light of the above teachings or may be acquired from practice of the present invention. The embodiments were chosen and described in order to explain the principles of the present invention and its practical application to enable one skilled in the art to utilize the present invention in various embodiments, and with various modifications, as are suited to the particular use contemplated. For example, the present invention could be utilized in conjunction with stationary audio players or could utilize various types of earpieces, including those having additional functionality such as microphones.

What is claimed is:

1. A method for controlling an electronic device capable of playing audio content via an earpiece, the method comprising:
receiving a signal representative of the state of the earpiece from the earpiece;
determining the state of the earpiece based upon the received signal; and
modifying operation of the electronic device in view of the determined state of the earpiece.

2. The method of claim 1, wherein the state of the earpiece is either in use or not in use.

3. The method of claim 2, wherein modifying operation includes:
if the state of the earpiece is in use, allowing the audio content to play; and
if the state of the earpiece is not in use, ceasing the audio content.

4. The method of claim 1, wherein the receiving of the signal occurs via a wire electrically connecting the earpiece to the electronic device.

5. The method of claim 1, wherein receiving the signal occurs via a wireless connection between the earpiece and the electronic device.

6. The method of claim 1, wherein determining the state of the earpiece includes determining if a temperature of a portion of the earpiece is within a predetermined range of temperatures.

7. The method of claim 6, wherein determining the state of the earpiece further includes determining motion characteristics of the earpiece.

8. The method of claim 1, wherein determining the state of the earpiece includes completing a circuit when the user wears the earpiece.

9. A computer program product, comprising:
computer code for receiving a signal representative of the state of the earpiece from the earpiece to the electronic device;
computer code for determining the state of the earpiece based upon the transmitted signal; and
computer code for modifying operation of the electronic device in view of the determined state of the earpiece.

10. The computer program product of claim 9, wherein the computer code for determining the usage state includes computer code for determining if a sample temperature is within a predetermined range.

11. The computer program product of claim 10, further comprising computer code for operation an application control interface for facilitating communication between the earpiece and the electronic device.

12. An earpiece module for controlling an electronic device, the module comprising:
an earpiece having a communications apparatus for communication with an electronic device;
an earpiece state sensor adapted to output a signal in response to a change in the state of the earpiece.

13. The earpiece module of claim 12, wherein the state sensor includes a temperature sensor.

14. The earpiece module of claim 12, wherein the state sensor includes a mechanical switched activated when the earpiece is in use.

15. An electronic device, comprising:
a processor for processing information;
a memory unit operatively connected to the processor;
a mobile audio device for playing an audio content and operatively connected with the processor; and
an earpiece device operatively connected with the processor and having a sensor for detecting a usage state of the earpiece device and outputting a signal containing information regarding the usage state,
wherein the memory unit includes:
computer code for recognizing the signal's information regarding the usage state; and
computer code for controlling the mobile audio device based upon the signal.

16. The electronic device of claim 15, wherein the electronic device includes a telephone.

17. The electronic device of claim 15, wherein the sensor outputs an in use signal when the sensor detects the earpiece changes from a not in use to an in use state, and wherein the sensor outputs a not in use signal when the sensor detects the earpiece changes from an in use state to a not in use state.

18. The electronic device of claim 17, wherein the computer code for controlling the mobile audio device further includes computer code for ceasing audio play when a not in use signal is recognized.

19. The electronic device of claim 18, wherein the memory unit further comprising computer code for storing a location of the audio content when audio play is ceased.

20. The electronic device of claim 19, wherein the computer code for controlling the mobile audio device includes computer code for resuming audio play when a in use signal is recognized, the audio play being resumed at the location where the audio content was ceased.
